(19) United States
(12) Patent Application Publication
(10) Pub. No.: US 2012/0253285 A1
(43) Pub. Date: Oct. 4, 2012

(54) Cannula Seal

(75) Inventors: Michael Patton, Austin, TX (US); Richard Mazzolla, Austin, TX (US); Michele Jen, Stafford, TX (US)

(73) Assignee: PATTON SURGICAL CORPORATION, Austin, TX (US)

(21) Appl. No.: 13/479,023

(22) Filed: May 23, 2012

Related U.S. Application Data
(62) Division of application No. 12/604,961, filed on Oct. 23, 2009, now abandoned.

(51) Int. Cl. A61B 17/34

(52) U.S. Cl. 604/167.01

(57) ABSTRACT

Trocar seal assemblies are disclosed which consist of a helically-shaped seal positioned within a trocar's cannula; wherein, as an instrument is inserted through the central bore of the helically-shaped seal, the helically-shaped seal expands, thereby increasing the diameter of the central bore. In other embodiments, a protective member is positioned within a retaining member; wherein the protective member is configured as a helix and, as an instrument is inserted through the central bore of the helix, the helix expands, thereby increasing the diameter of the central bore.

### CROSS-REFERENCE TO RELATED APPLICATION

The present application is a divisional application of U.S. patent application Ser. No. 12/604,961 filed Oct. 23, 2009 in the name of Michael Patton, Richard Mazzolla and Michele Jen, entitled “Cannula Seal,” which claims priority from and incorporates by reference prior U.S. Provisional Patent Application No. 61/107,850, filed Oct. 23, 2008.

### BACKGROUND OF THE INVENTION

Laparoscopy is a minimally invasive surgical procedure that utilizes a small tubular camera (laparoscope) to view abdominal and pelvic organs. During laparoscopy, devices called “trocars” are used to puncture the abdominal wall and provide access channels for the camera and thin laparoscopic surgical instruments. Since smaller incisions are made during these procedures, there is less patient trauma and reduced hospitalization. As a result, laparoscopy continues to grow in popularity.

A trocar assembly generally includes two major components, an obturator and a cannula. The obturator typically includes an elongate body having a sharpened distal tip. The sharp distal tip pierces and cuts the tissue forming the body wall. The cannula generally has a cylindrical configuration and a seal-valve housing. As the trocar is pushed or otherwise moved through the body wall, the sharp distal tip of the obturator functions to cut the tissue and provide an opening for the trocar. Once the trocar is operatively positioned, the obturator can be removed leaving the cannula to provide working access into the body cavity. For example, a laparoscope may be inserted through the cannula to view the body cavity or surgical instruments may be inserted through the cannula to perform ligations or other procedures.

The use of cannulas in laparoscopic surgery is well known. Once initial access to the abdominal cavity is attained, it is filled with carbon dioxide gas to allow for optimal viewing with the laparoscope and room for instrument manipulation. The cannula, which maintains the incision open to receive surgical instruments, must have a sealing mechanism that prevents or limits the escape of the gas when instruments of various diameters are inside. The present invention provides a novel sealing system that overcomes deficiencies in sealing systems currently known in the art.

### SUMMARY OF THE INVENTION

The present invention provides an improved cannula seal for use in a wide variety of surgical procedures and is intended to allow instruments of various diameters to be repeatedly inserted and removed, through a single cannula, without rupturing or otherwise diminishing the integrity of the seal, without excessive drag on the instruments, and without the need for lubricants or slip agents. While the seal of the present invention is described herein for use with a surgical trocar, it is also well suited for a variety of other uses in which one apparatus is inserted and removed through an opening.

In one embodiment, the seal assembly consists of a protective member and a retaining member. The protective member prevents accidental perforations of the retaining member during the insertion of an instrument through the seal assembly and assists in forming a seal against the instrument when inserted in the seal assembly. The protective member is generally configured with a circular upper portion a generally conical lower portion. The lower portion of the protective member consists of a plurality of flaps which increasingly overlap as they progress to the lower portion of the conical shape, thereby forming a seal at the lowest portion where the flaps overlap entirely. The overlapping flaps expand with minimal resistance when instruments are inserted through the seal assembly, thereby ensuring a proper seal regardless of the size or configuration of the instrument. The retaining member holds the protective member in position and compresses the protective member against an instrument when an instrument is inserted through the seal assembly.

In an alternative embodiment, a guide member is positioned within the retaining member to form the seal assembly. The guide member guides the instrument into the seal assembly so that the instrument does not damage the retaining member. The guide member can be configured with a circular upper portion and a generally conical lower portion which includes a plurality of grooves which direct the instrument toward the opening at the distal end of the guide member.

In another embodiment, the protective member may be configured helically, without flaps. As the helical shape unwinds, the circumference of the inner bore expands, thereby decreasing resistance on instruments as they are inserted and withdrawn from the protective member.

The foregoing has outlined rather broadly certain aspects of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### DESCRIPTION OF THE DRAWINGS

For a more complete understanding of the present invention, and the advantages thereof, reference is now made to the following descriptions taken in conjunction with the accompanying drawings, in which:

FIG. 1 is a cross-sectional view of a cannula with one embodiment of the seal assembly of the present invention positioned therein;

FIG. 2 is an cross-section view of the proximal end of a cannula with one embodiment of the seal assembly of the present invention in a closed position;

FIG. 3 is a cross-section view of the proximal end of a cannula with one embodiment of the seal assembly of the present invention with an instrument positioned therethrough;

FIG. 4A is a side view of one embodiment of the protective member of the present invention in the closed position;

FIG. 4B is a bottom or distal view of one embodiment of the protective member of the present invention in the closed position;

FIG. 5 is top view of one embodiment of the protective member with folds in an open position;

FIG. 6 is an isometric view of one embodiment of the protective member with folds in an open position;

FIG. 7 is an isometric view of one embodiment of the seal assembly of the present invention with an instrument inserted therethrough;

FIG. 8 is cross-sectional view of the proximal end of a cannula with one embodiment of the seal assembly of the present invention with a guide member positioned therein;

FIG. 9 is a cross-sectional view of the proximal end of a cannula with one embodiment of the seal assembly of the present invention with a protective member and a guide member positioned therein;

FIG. 10A is a top view of one embodiment of the guide member of the present invention;

FIG. 10B is an isometric view of one embodiment of the guide member of the present invention;

FIG. 10C is another isometric view of one embodiment of the guide member of the present invention;

FIG. 11A is a side view of another embodiment of the seal assembly of the present invention;

FIG. 11B is a top view of another embodiment of the seal assembly of the present invention; and

FIG. 12 is an exploded view of another embodiment of the seal assembly of the present invention.

### DESCRIPTION OF THE PREFERRED EMBODIMENTS

The present invention is directed to a seal assembly for sealed reception of an elongated object, such as a surgical instrument, through an aperture, where the seal assembly selectively opens and closes the aperture to permit entry of the object therethrough. The configuration and using of the presently preferred embodiments are discussed in detail below. It should be appreciated, however, that the present invention provides many applicable inventive concepts that can be embodied in a wide variety of contexts other than as a trocar seal. For example, while the seal of the present invention is described for use in a surgical cannula, it may similarly be useful in pumps, heat exchangers, and numerous other applications. Accordingly, the specific embodiments discussed are merely illustrative of specific ways to make and use the invention, and do not limit the scope of the invention. In addition, references to surgical procedures and other terms used herein may be applicable to medical and veterinary surgery and, when the seal assembly is used in connection with surgical trocars, references to instruments or other devices that may be inserted through the cannula include clip appliers, dissectors, graspers, laser and electrocautery devices, drainage or fluid introduction tubes and other types of surgical instruments.

The present invention provides an improved seal assembly for use with a cannula during surgery. In one embodiment, the seal assembly consists of a protective member and a retaining member. The protective member prevents accidental perforations of the retaining member during the insertion of an instrument through the seal assembly and assists in forming a seal against the instrument when inserted in the seal assembly. The protective member is generally configured with a circular upper portion a generally conical lower portion. The lower portion of the protective member consists of a plurality of flaps which increasingly overlap as they progress to the lower portion of the conical shape, thereby forming a seal at the lowest portion where the flaps overlap entirely. The overlapping flaps expand with minimal resistance when instruments are inserted through the seal assembly, thereby ensuring a proper seal regardless of the size or configuration of the instrument. More specifically, the flaps expand so that the opening accommodates the diameter of the instrument and then the flaps form a seal around the instrument. The retaining member holds the protective member in position and compresses the protective member against an instrument when an instrument is inserted through the seal assembly.

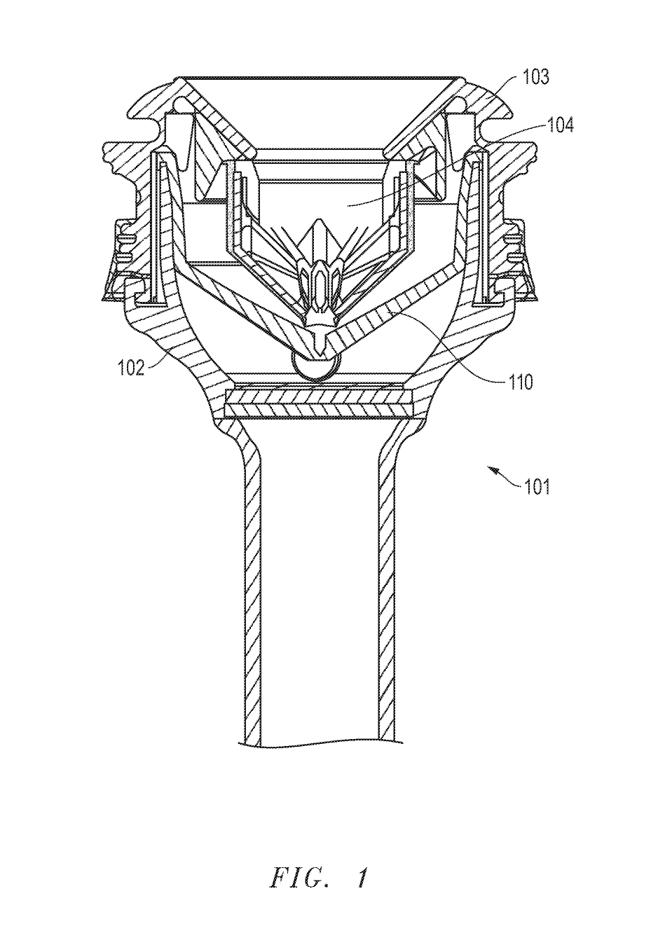

Referring now to the drawings, FIG. 1 shows a cannula 101 with a cannula body 102 and a removable cannula cap 103. A seal assembly 104 is removably positioned within the cannula cap 103. A valve 110 is positioned on distal side of the seal assembly 104 to keep the carbon dioxide from escaping when an instrument is not positioned in the cannula 101.

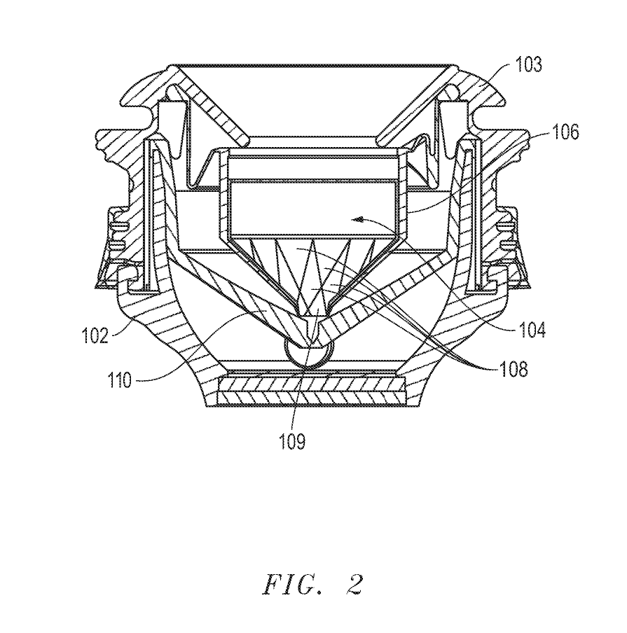

FIG. 2 shows one embodiment of the seal assembly 104 when it is not penetrated by an instrument. In this embodiment, the seal assembly 104 consists of a protective member 105 and a retaining member 106. Although the protective member 105 and the retaining member 106 are shown in this embodiment as separate components of the seal assembly 104, they can be a single unit made of one material, a single unit made of laminated materials with different properties, or any other configuration to achieve the desired result.

A protective member 105 prevents accidental perforations of the retaining member 106 during the insertion of an instrument through the seal assembly 104 and assists in forming a seal against the instrument when inserted in the seal assembly 104. The protective member 105 can be made of any tear-resistant material such as, for example, monofilament mesh of an open or closed weave, stainless steel mesh, or polymer sheet, and the thickness of the material will depend on the nature of the application. The protective member 105 is configured with a circular upper portion 107 and a generally conical lower portion. The lower portion of the protective member 105 consists of a plurality of flaps 108 which increasingly overlap as they progress to the lower portion 109 of the conical shape, thereby forming a seal at the lowest portion 109 where the flaps 108 overlap entirely. The overlapping flaps 108 expand with minimal resistance when instruments are inserted through the seal assembly 104, thereby ensuring a proper seal regardless of the size or configuration of the instrument.

The retaining member 106 holds the protective member 105 in position and compresses the protective member 105 against an instrument when an instrument is inserted through the seal assembly 104. The retaining member 106 can be made of any suitable material known in the art, such a polymeric, silicone or rubber material or a composite. By compressing the protective member 105 against an instrument during use, the seal assembly 104 creates a seal around the instrument to effectively prevent the leakage of gas through the seal assembly 104. In other embodiments of the invention, the retaining member can be used either without a protective member 105 of any kind, or with a protective member where the lower portion 109 of the flaps 108 of the protective member 105 do not extend distally beyond the distal end of the retaining member 106. In these cases, the retaining member 106 itself can form a seal around the instrument to effectively prevent the leakage of gas through the seal assembly 104

Referring now to FIG. 3 in which an instrument 111 has been inserted through one embodiment of the seal assembly 104. When the instrument 111 is first inserted into the seal assembly 104, it passes through the upper portion 107 of the protective member 105 and contacts the flaps 108, thereby causing the flaps 108 to expand and open to allow the instrument 111 to pass through. Because the instrument 111 first contacts the protective member 105 and not the retaining member 106, the retaining member 106 is not damaged by the instrument 111 as it passes through the seal assembly 104. As previously stated, the overlapping flaps 108 expand with minimal resistance when the instrument 111 is inserted through the seal assembly 104, thereby ensuring that a proper seal forms around the instrument 111. The retaining member 106 holds the protective member 105 in position and compresses the protective member 105 against the instrument 111 in the compression zone 112. In one embodiment the distal portion 113 of the flaps 108 extend past the lower portion of the retaining member 106 to ensure that an adequate seal is formed within the compression zone 112. By compressing the protective member 105 against the instrument 111, the seal assembly 104 creates a seal around the instrument 111 at the compression zone 112, thereby preventing the leakage of gas through the seal assembly 104.

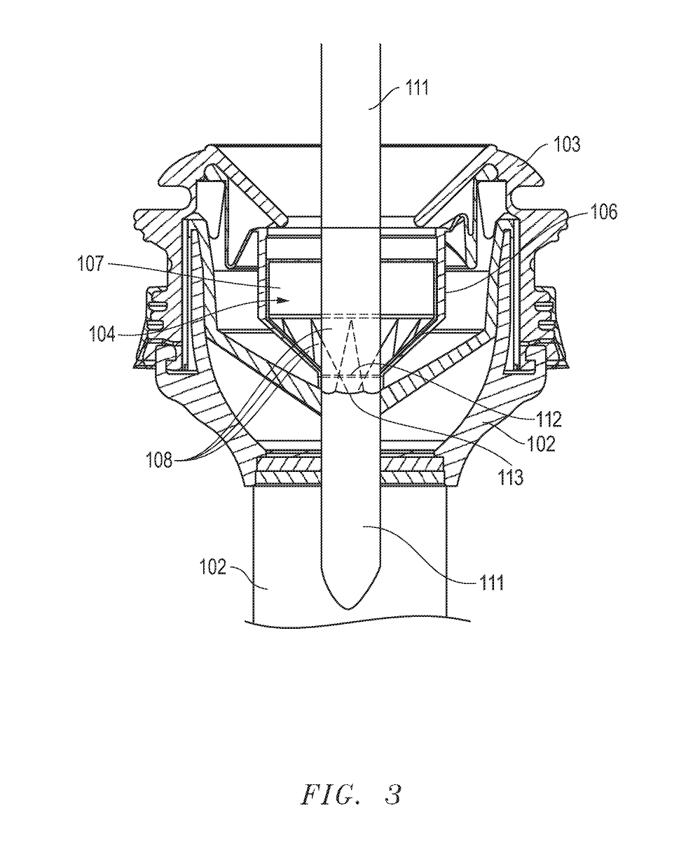

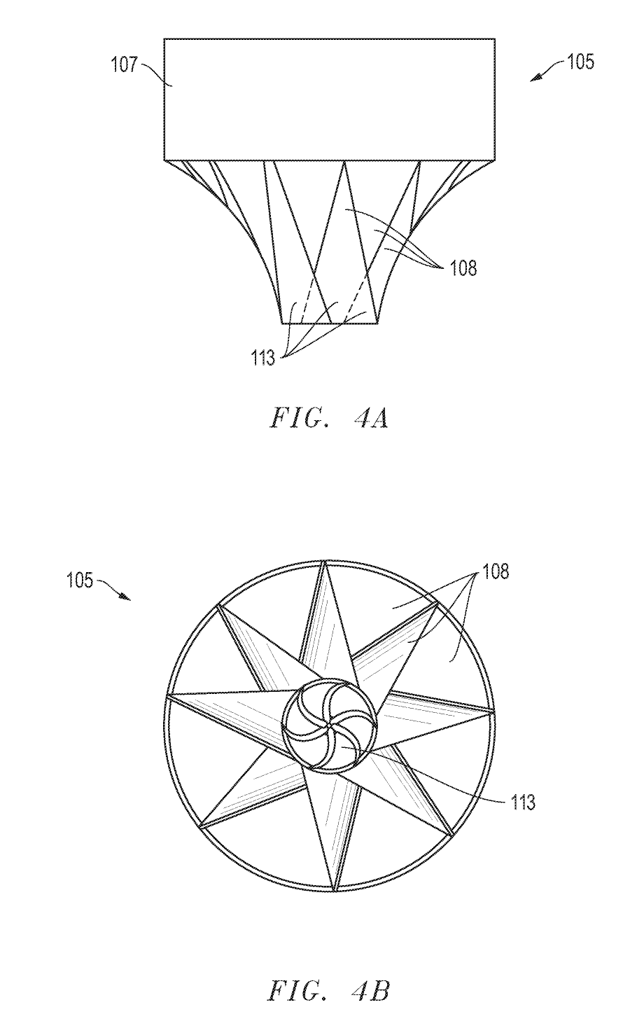

FIG. 4A shows a side view of one embodiment of the protective member 105 without an instrument inserted therethrough. The upper portion 107 is generally circular and can be any diameter required to fit within the retaining member 106, which, in turn, is configured to fit within the trocar cap 103. The height of the upper portion 107 will also be determined by the constraints of the retaining member 106 and the trocar cap 103. The plurality of flaps 108 which make up the lower portion of the protective member 105 can be any configuration suitable for the application and need not be configured in a triangular as depicted in FIG. 4A. The distal portion 113 of the flaps 108 are generally aligned so that one flap 108, or portion thereof, does not extend beyond the others.

FIG. 4B shows a bottom or distal view of one embodiment of the protective member 105 wherein one configuration of the flaps 108 is depicted. In this configuration, the flaps 108 increasingly overlap as they progress to their distal portion 113, thereby forming a seal where the flaps 108 overlap entirely.

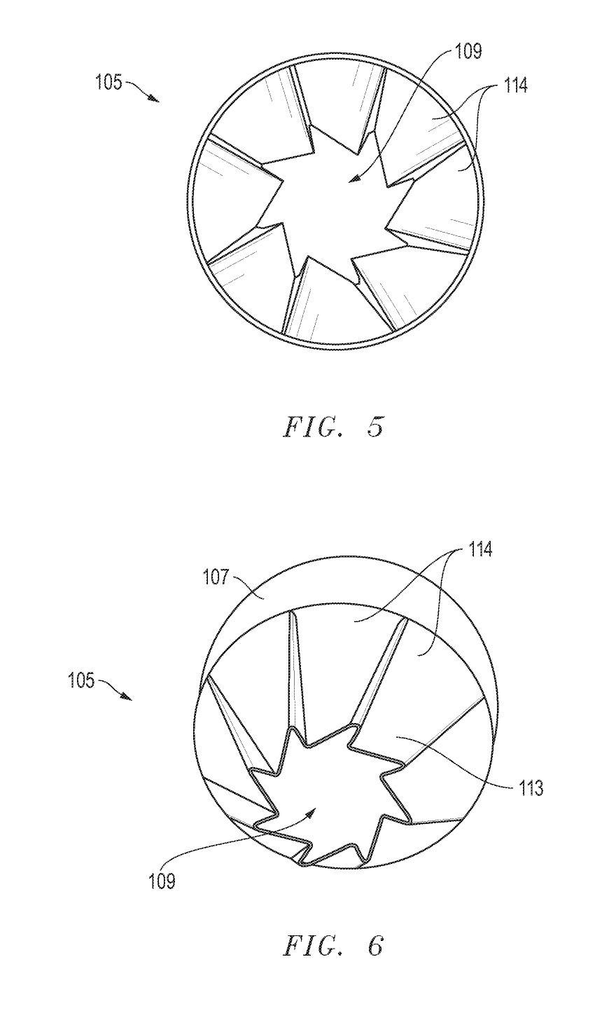

FIG. 5 shows a top view of one embodiment of the protective member 105, and FIG. 6 shows a corresponding isometric view of one embodiment of the protective member 105, in the configuration it would appear with an instrument inserted therethrough, In this configuration, the protective member 105 is configured with folds 114 instead of flaps 108. The plurality of folds 114 which make up the lower portion of the protective member 105 are extended such that an opening 109 appears at the distal end.

FIG. 7 shows an instrument 111 inserted through another embodiment of the seal assembly 104. In this embodiment, the protective member 105 and the retaining member 106 are combined to create a single-member seal. As with previously described seal assemblies, the overlapping flaps 108 in this embodiment expand with minimal resistance when the instrument 111 is inserted therethrough, thereby ensuring that a proper seal forms around the instrument 111 in the compression zone 112. The distal portion 113 of the flaps 108 ensure that an adequate seal is formed within the compression zone 112.

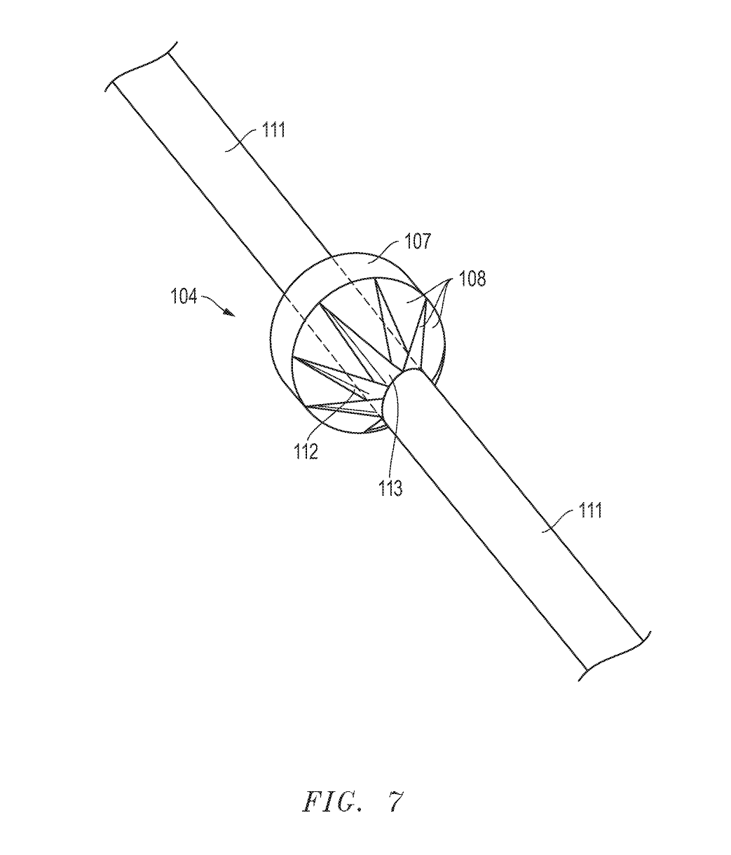

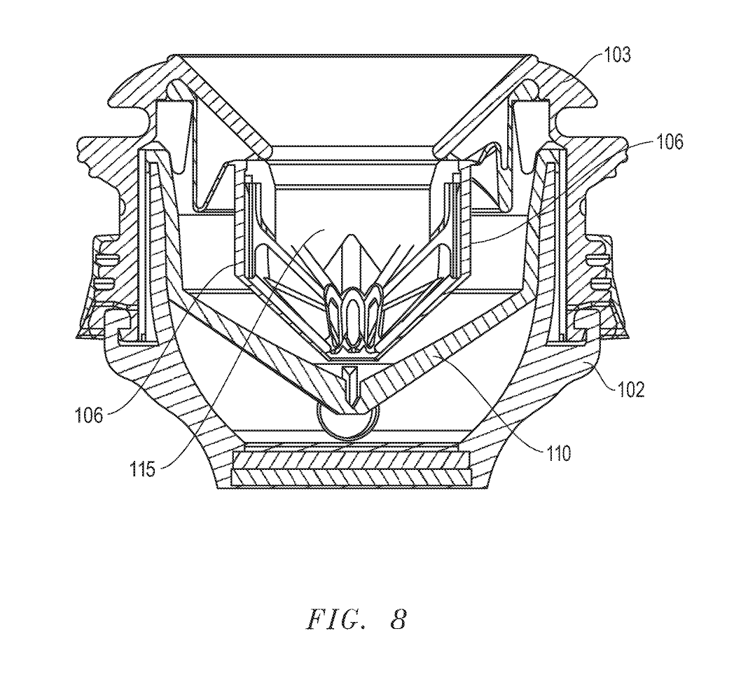

Referring now to FIG. 8 in which a guide member 115 is positioned within the retaining member 106. Once again, the retaining member 106 is positioned within a cannula cap 103 which is removably affixed to the cannula body 102. Valve 110 is positioned on the distal side of the retaining member 106 to keep the carbon dioxide from escaping when an instrument is not positioned in the cannula 101. In this embodiment, the seal assembly 104 consists of a guide member 115 and a retaining member 106. The guide member 115 guides an instrument into the seal assembly 104 so that the instrument does not damage the retaining member 106. The guide member 115 can be made of any material that is sufficiently rigid to guide the instrument through the seal assembly 104 while protecting the retaining member 106 from damage.

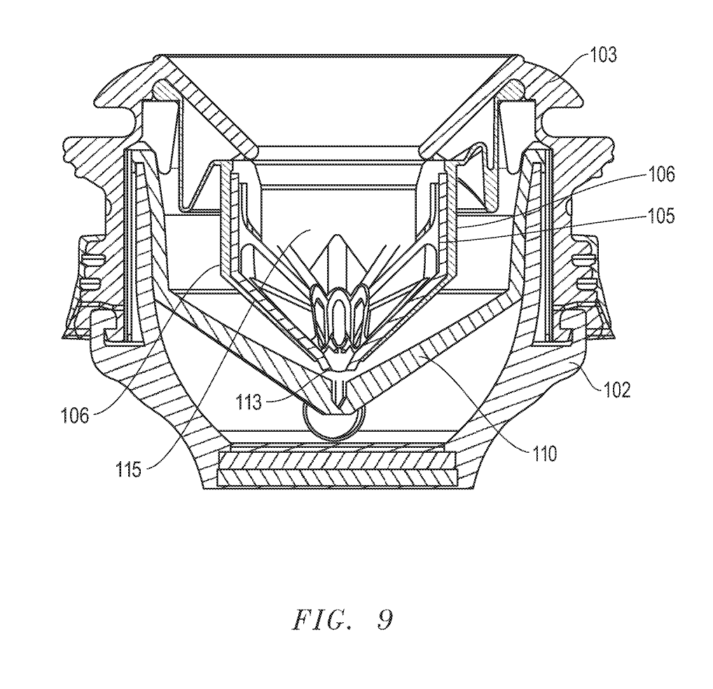

FIG. 9 shows an alternative embodiment of the present invention which also incorporates a guide member 115. In this embodiment, the seal assembly 104 consists of a guide member 115, a retaining member 106, and a protective member 105. The retaining member 106 is positioned within a cannula cap 103 which is removably affixed to the cannula body 102. The protective member 105 is positioned inside the retaining member 106 and the guide member 115 is positioned within the protective member 105. Once again, the guide member 115 guides an instrument into the seal assembly 104 so that the instrument does not damage the retaining member 106. The retaining member 106 holds the protective member 105 in position and compresses the protective member 105 against an instrument when an instrument is inserted through the seal assembly 104. By compressing the protective member 105 against an instrument during use, the seal assembly 104 creates a seal around the instrument to effectively prevent the leakage of gas through the seal assembly 104.

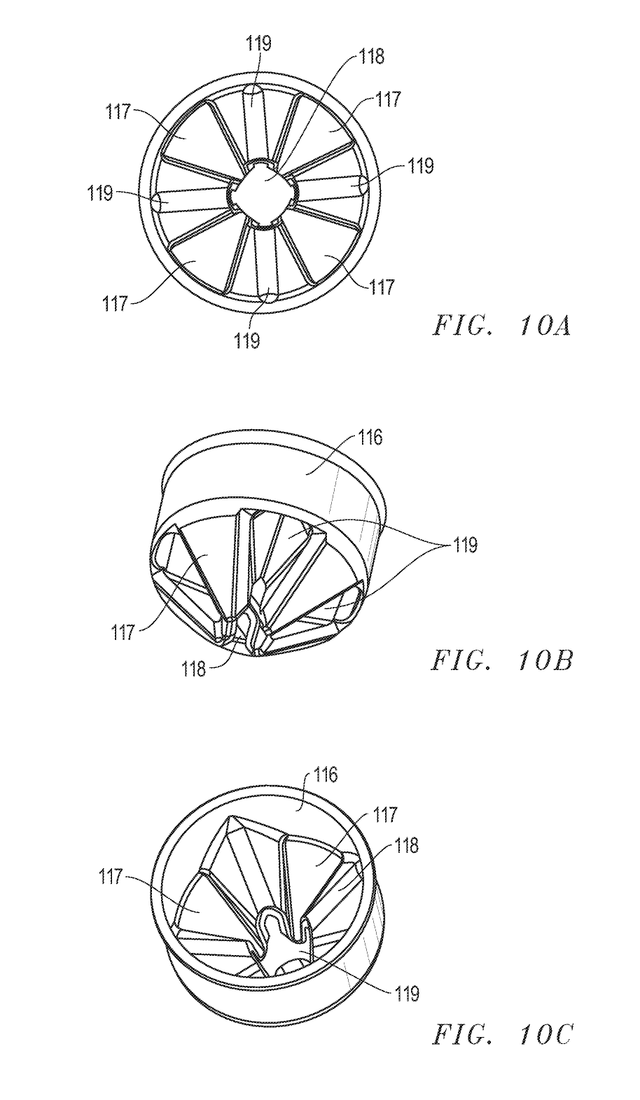

The guide member 115 can be any configuration suitable for guiding the instrument through the seal assembly 104. FIG. 10A, FIG. 10B and FIG. 10C show a top view, an isometric view of the distal end and an isometric view of the proximal end, respectively, of one embodiment of the guide member 115.

In this embodiment, the guide member 115 is configured with a circular upper portion 116 and a generally conical lower portion which includes a plurality of grooves 117 and ridges 119. When an instrument is inserted into the guide member 115, the instrument first contacts the ridges 119 whereupon the ridges 119 collapse downwards towards the grooves 117, thereby expanding the diameter of the opening 118. As the instrument proceeds through the opening 118, the distal portions of the guide member 115 form a seal around the instrument. As a result, the instrument is efficiently guided through the guide member 115 while the retaining member 106 remains protected. The guide member 115 can be made of any material rigid enough to guide the instruments toward the opening 118 and to protect the retaining member 106 from damage by the instrument while still being pliable enough to form a seal around an instrument inserted through the opening 118.

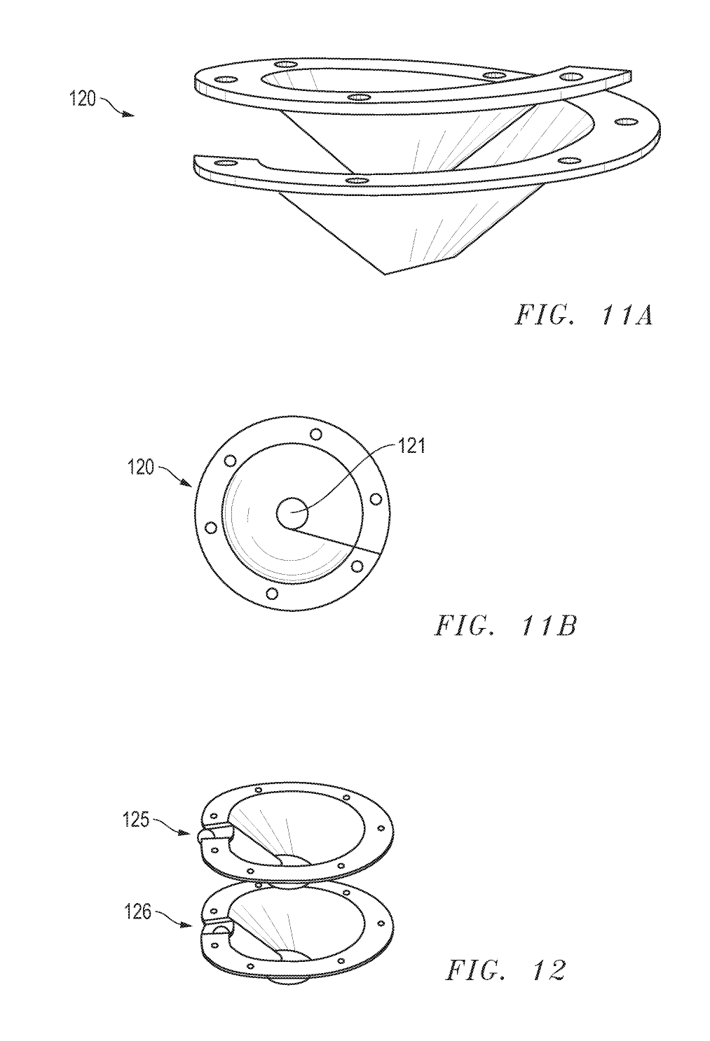

As previously described, protective member 105 may appear in a variety of embodiments. One alternative embodiment is shown in FIG. 11A and FIG. 11B. FIG. 11A shows a side view of an alternative protective member 120 which is made without flaps, but which is instead configured conically as a helix. The helical shape increases the circumference of the inner bore 121, thereby decreasing resistance on instruments as they are inserted and withdrawn from the protective member 120. As an instrument is inserted through the protective member 120, the circumference of the inner bore 121 expands as the helix unwinds. The inner bore 121 expands just enough to allow the instrument pass through and, because the protective member 120 is formed of a pliable material, the inner bore adheres to the instrument sufficiently to form a seal.

FIG. 12 shows an exploded view of another embodiment of the present invention in which a seal assembly consists of a protective member 125 which is positioned within a retaining member 126. Both the protective member 125 and the retaining member 126 are helically shaped in a configuration substantially similar to that shown in FIG. 11A and FIG. 11B. The protective member 125 can be made of any tear-resistant material such as, for example, monofilament mesh of an open or closed weave, stainless steel mesh, polymer sheet and the thickness of the material will depend on the nature of the application. The retaining member 126 can be made of any suitable material known in the art, such a polymeric, silicone or rubber material or composite. In this embodiment, as previously disclosed, as the helical shape unwinds, the circumference of the inner bore expands, thereby decreasing resistance on instruments as they are inserted and withdrawn from the protective member 120.

While the present system and method has been disclosed according to the preferred embodiment of the invention, those of ordinary skill in the art will understand that other embodiments have also been enabled. Even though the foregoing discussion has focused on particular embodiments, it is understood that other configurations are contemplated. In particular, even though the expressions “in one embodiment” or “in another embodiment” are used herein, these phrases are meant to generally reference embodiment possibilities and are not intended to limit the invention to those particular embodiment configurations. These terms may reference the same or different embodiments, and unless indicated otherwise, are combinable into aggregate embodiments. The terms “a”, “an” and “the” mean “one or more” unless expressly specified otherwise. The term “connected” means “communicatively connected” unless otherwise defined.

When a single embodiment is described herein, it will be readily apparent that more than one embodiment may be used in place of a single embodiment. Similarly, where more than one embodiment is described herein, it will be readily apparent that a single embodiment may be substituted for that one device.

In light of the wide variety of possible seals available, the detailed embodiments are intended to be illustrative only and should not be taken as limiting the scope of the invention. Rather, what is claimed as the invention is all such modifications as may come within the spirit and scope of the following claims and equivalents thereto.

None of the description in this specification should be read as implying that any particular element, step or function is an essential element which must be included in the claim scope. The scope of the patented subject matter is defined only by the allowed claims and their equivalents. Unless explicitly recited, other aspects of the present invention as described in this specification do not limit the scope of the claims.

What is claimed is:

1. A trocar seal comprising:
a helically-shaped seal positioned within a trocar's cannula; wherein, as an instrument is inserted through the central bore of said helically-shaped seal, said helically-shaped seal expands, thereby increasing the diameter of said central bore.

2. A seal assembly comprising:
a protective member positioned within a retaining member; wherein said protective member is configured as a helix and, as an instrument is inserted through the central bore of said helix, the helix expands, thereby increasing the diameter of said central bore.

3. The seal assembly of claim 2, wherein said central bore of said helix compresses against said instrument to form a seal.

4. The seal assembly of claim 2, wherein said retaining member compresses directly against said instrument to form a seal.

5. A seal assembly comprising:
a protective member positioned within a retaining member; wherein said protective member is configured as a helix and, as an instrument is inserted through the central bore of said helix, the helix expands, thereby increasing the diameter of said central bore; and wherein said retaining member is also configured as a helix.

6. The seal assembly of claim 5, wherein said central bore of said protective member compresses against said instrument to form a seal.

7. The seal assembly of claim 5, wherein said retaining member compresses directly against said instrument to form a seal.
